(19) United States
(12) Patent Application Publication
(10) Pub. No.: US 2010/0004677 A1
(43) Pub. Date: Jan. 7, 2010

(54) SHAFTED SURGICAL INSTRUMENTS FOR REMOTE ACCESS SURGICAL PROCEDURES

(75) Inventors: Daniel Brostoff, Aliso Viejo, CA (US); Franz Jakoubek, Liptingen (DE)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/493,977

(22) Filed: Jun. 29, 2009

Related U.S. Application Data
(63) Continuation-in-part of application No. 29/335,238, filed on Apr. 10, 2009.
(60) Provisional application No. 61/078,184, filed on Jul. 3, 2008.

(51) Int. Cl. A61B 17/29

(52) U.S. Cl. 606/205

(57) ABSTRACT

An instrument for endoscopic minimally invasive procedures has a shaft with a working end, such as a clamp, scissors, forceps, or the like. Handle portions extend in an opposed relationship and pivot relative to the shaft. When the handle portions are moved about respective pivot points, the working end of the surgical instrument is actuated. The first and second handle portions each have an indented grasping segment in which a surgeon's finger or thumb may rest. The grasping segments have rounded cross-sections to facilitate a surgeon rotating the handle with a finger and another finger or thumb. The indented portions form a narrow portion when the handle is in a closed configuration. There may be a 1:1 ratio between handle movement and that of the working end, and the balance point of the instrument may be at the distal end of the handle.

### I. CROSS-REFERENCE TO RELATED APPLICATIONS

This application claims priority from U.S. Provisional Patent Application No. 61/078,184, filed on Jul. 3, 2008 and entitled “Ergonomic Handle for Shafted Instruments,” and is a continuation-in-part of US Design Patent Application No. 29/335,238, filed on Apr. 10, 2009, both of which are hereby incorporated by reference in their entirety.

### II. BACKGROUND

Many surgical instruments include a working end or effector, such as a clamp, scissors, forceps, needle holder, graspers, pusher, etc. that is connected to a central shaft. Distal from the working end is typically a handle for grasping and manipulating the instrument. In many instances, the handle includes an actuating mechanism that is linked to and that actuates the working end or effector. For example, scissors may include blade portions that are moveable relative to each other. The scissors are connected to a shaft extending away from the scissors. At the distal end, the handle includes a linkage that can be manipulated by a user to actuate the scissors.

U.S. Pat. No. 5,501,698 discloses a device for performing minimally-invasive microsurgery such as thoracoscopic coronary artery bypass grafting. The instrument generally includes a pair of coaxially arranged shafts, an end-effector at the distal ends of the shafts, and an actuator at the proximal ends of the shafts. The actuator includes a pair of generally straight arms pivotally coupled to a shaft. The links are coupled to a proximal portion of the arms for mechanical advantage. The instruments may be provided in a variety of sizes, depending on the particular purpose for which the instrument is to be used.

### III. SUMMARY OF THE INVENTION

The present invention provides an instrument for use in intricate, minimally-invasive procedures. The instrument has an ergonomic handle with one or more of various features providing advantages over existing prior art handles. For example, in one embodiment, the handle of the present invention may be formed at least partially of titanium or another metal that is resistive to corrosion during cleaning of the handle. Titanium may provide an added benefit of decreasing the overall weight of the handle.

In some embodiments, material at the center portion of the handle may be removed to provide an open handle configuration. The handle portions may thus have a reduced side profile. This embodiment reduces the surface area of the handle that may harbor bacteria, dirt, etc. It may also reduce areas that would be difficult to clean.

The handle may include a gripping section for frictionally engaging the user's hand. In an additional or alternative embodiment, the handle includes a contoured section that increases the ergonomic aspects of the handle. The handle may also create a decreased number of connections to the surgical instrument, thereby decreasing the instrument's complexity and possible failure points. In addition, the connection design of the handle to the surgical instrument may also for increased control of the working end of the surgical instrument via the handle.

The handle typically includes two or more handle portions in an opposed relationship that are connected to the surgical instrument. The handle portions may extend from an end of the shaft in a direction toward the working end of the instrument, whereby the handle portions overly a portion of the shaft, such that the handle either does not or only minimally extends past the end of the surgical instrument. In this embodiment, the handle is in a compact configuration reducing the overall length of the instrument.

In some embodiments, the handle portions may provide clearance relative to the shaft so as increase the motion of the handle portions relative to each other, thereby providing more precise control of the surgical instrument. The handle may also allow the surgical instrument to be more easily rotated in the hands of the user without the need to remove the surgical instrument from the surgical site to be repositioned in the user's hand.

Considering specific exemplary combinations of inventive features, one embodiment relates to a surgical instrument having a shaft and a handle. A rod is located in the shaft and extends between a first end that is connected to a working end of the surgical instrument, and a second end that connects to the handle. The first and second handle portions are in pivotal communication with the shaft. First and second linkages respectively connect the first and second handle portions to the rod at or near the distal end of the rod. When the first and second handles are moved about respective pivot points relative to the shaft, the rod operates the working end of the surgical instrument. To accommodate a finger and another finger or thumb of the surgeon in an advantageous manner, the handle portions may each have an indented grasping segment, typically at the distal end region of the handle.

Additional features may be included. For instance, the indented handle areas may have a rounded cross-section to facilitate a surgeon rotating the handle. The cross-section may be, for example, semi-elliptical. The indented portions may also include gripping surfaces with ridges to assist in firmly grasping the handle. The indented portions may also each include a finger stop at a distal end thereof to permit the surgeon to easily grasp the handle in a preferred location.

Typically, the handle will have an open configuration and a closed configuration. The indented portions together form a narrow handle portion when the handle is in the closed configuration. In one embodiment, the handle at a widest portion is at least two times wider than the handle at a narrow portion (e.g. in the indented region) when the handle is in a closed configuration. Another feature is that the handle at a widest portion may be at least four times wider than the shaft, in the closed configuration, to provide rotational stability.

The handle may include walls having elongated openings to facilitate cleaning of the handle. The handle may also be configured to have a 1:1 movement ratio between movement of the handle by the surgeon, as when the surgeon is pressing down on the handle to move the working end into a closed position, and the corresponding movement of the working end. In a preferred embodiment, the indentation depth ratio of the handle is between 0.35 and 0.55; and the indentation length ratio of the handle is between 0.65 and 0.85

In any of the specific embodiments, the handle of the surgical instrument may be compact relative to the overall length of the device, with the handle being no more than approximately one third as long as the total length of the surgical instrument. The balance point of the instrument may be at approximately the distal end of the handle. In some embodiments, the balance point is at approximately ⅓ of the length of the instrument from the proximal end of the instrument.

The handle of any of the embodiments may have a two-portion structure, with a relatively heavy proximal portion and a lighter distal portion that curves inwardly sharply and that has a rounded cross-section. The relatively heavy proximal portion acts as a counterweight, so that there is a balance point at about the point where the surgeon is grasping the device. The counterweight makes the device more stable. The proximal portion of the handle may also be relatively wider than the distal portion when the handle is in a closed configuration, as described above, to add rotational stability.

The aforementioned features may be combined in a variety of different ways to form different embodiments. It should also be understood that the foregoing Summary is not a complete description of the inventive features and aspects of the invention. Other features and advantages of the present invention will become more apparent from the following Detailed Description, taken in conjunction with the drawings, and from the claims.

### IV. BRIEF DESCRIPTION OF THE DRAWINGS

FIG. 1 is a perspective view of a scissors embodiment of the present invention in an open configuration;

FIG. 1A is a detail view of the scissors portion of FIG. 1;

FIG. 2A is a partial side view of the embodiment of FIG. 1 with the handle in a closed configuration;

FIG. 2B is a partial side view of the embodiment of FIG. 1 with the handle in an open configuration;

FIG. 2C is front view of the embodiment of FIG. 2A;

FIG. 2D is a side view illustrating dimensions of the handle of one embodiment of the present invention;

FIG. 3 is a perspective view of a gripper embodiment of the present invention, with the gripper and the handle in an open configuration;

FIG. 3A is a detail view of a locking mechanism to lock the handle in a closed position;

FIG. 4 illustrates the embodiment of FIG. 3 with the handle in a closed and locked configuration and with the gripper closed;

FIG. 5 is a front view of the configuration of FIG. 4;

FIG. 6 is an end view of the configuration of FIG. 4;

FIG. 7 is a side view of the configuration of FIG. 4 and showing the arms of the locking mechanism locked together; and

FIG. 8 is a perspective view of a surgeon's hand grasping an embodiment of the present invention, in which the balance point of the device is at the distal end of the handle.

### V. REFERENCE NUMBERS

The following table summarizes the reference numbers used in conjunction with the accompanying Figures:

| | |
|---|---|
| 10 | handle |
| 12 | surgical instrument |
| 14 | shaft |
| 16 | 1^{st} end of shaft |
| 18 | 2^{nd} end of shaft |
| 20 | cutting edges |
| 22 | instant housing |
| 24 | rod |
| 26 | flush port |
| 28 | interface connector |
| 30 | bias mechanism |
| 32 | end cap |
| 34 | spring |
| 36 | 1^{st} handle portion |
| 38 | 2^{nd} handle portion |
| 40 | pivot pin |
| 42 | 1^{st} linkage |
| 44 | 2^{nd} linkage |
| 46 | opening |
| 48 | outer contoured surface |
| 50 | contoured surface |
| 52 | narrow portion |
| 54 | narrow portions |
| 56 | gripping surface |
| 58 | contoured surface |
| 60 | arched or angled inner surface |
| 62 | arched or angled inner surface |
| 64 | locking mechanism |
| 66 | first lock |
| 66a | L-Shaped portion |
| 68 | second lock |
| 68a | L-Shaped portion |
| 70 | slot |
| 72 | slot |
| 74 | bias mechanism |
| 76 | bias mechanism |
| 78 | slot |
| 80 | surgeon's hand |
| 82 | distal end of handle portion |
| 84 | distal end of handle portion |
| D1 | length of handle |
| D2 | length of handle to gripping portion |
| D3 | length of handle to the linkage |
| D4 | width of one handle piece at the base |
| D5 | dimension at start of indented |
| | portion to the tip |
| D6 | width of end tip of handle |
| D7 | length of inwardly curving portion of |
| | the handle |
| D8 | width of tip of handle portion measured |
| | from narrowest portion of the handle |
| D9 | length of surgical instrument |
| D10 | width of handle at a wide portion |
| D11 | diameter of shaft |
| D12 | diameter of handle at a narrow portion |

### VI. DETAILED DESCRIPTION OF A PREFERRED EMBODIMENT

The following description of embodiments refers to the accompanying drawings, which illustrate specific embodiments of the invention. Other embodiments having different structures and operation do not depart from the scope of the present invention. For example, the handle of the present invention is illustrated herein in communication with different surgical instrument embodiments. It is understood that the handle of the present invention is not confined to the specific embodiments shown herein. These embodiments merely illustrate the concepts of the invention as applied to specific surgical instruments.

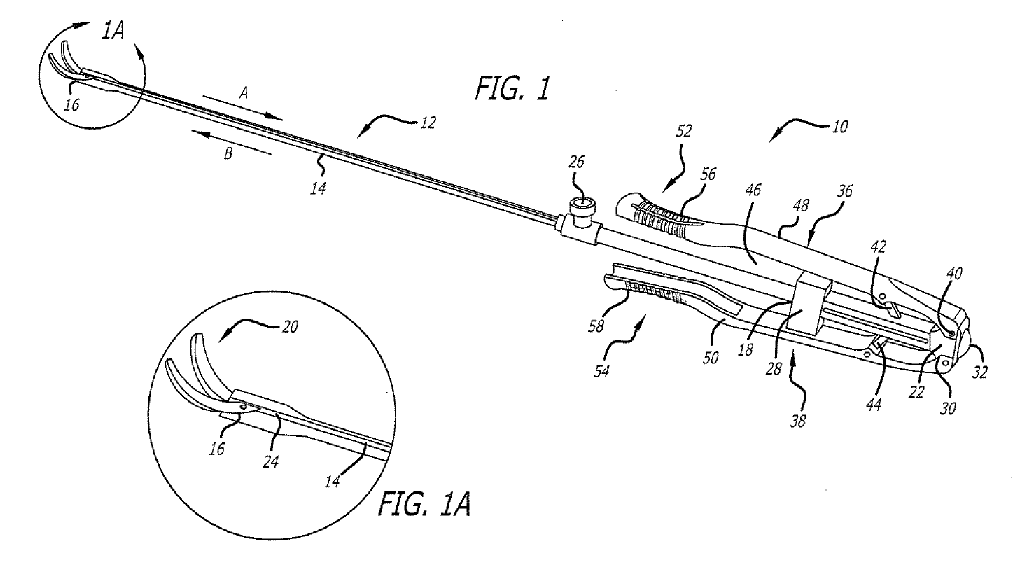

FIG. 1 illustrates a handle 10 according to one embodiment of the present invention. The handle is illustrated in communication with a surgical instrument 12. The depicted surgical instrument 12 includes a shaft 14 extending laterally between opposed first and second ends, respectively 16, 18. The first end 16 is a working end or effector for the surgical instrument 12. For example, in this embodiment, the working end 16 are scissors comprising a pair of cutting edges 20. The second end 18 of the surgical instrument 12 is connected to an instrument housing 22. While not specifically pertinent to the handle design, the surgical instrument may include a flush port 26 connected to the shaft for purposes of cleaning the instrument.

As illustrated in FIG. 1A, the cutting edges 20 of the working end 16 of the surgical instrument are actuated by a rod 24 located in the shaft 14. The rod 24 is connected to either one or both of the cutting edges 20, depending on the design of the surgical instrument. Movement of the rod in a lateral direction A away from the cutting edges 20 causes movement of the cutting edges toward each other in a cutting motion. And movement of the rod 24 in a lateral direction toward the cutting edges 20 causes the cutting edges to separate.

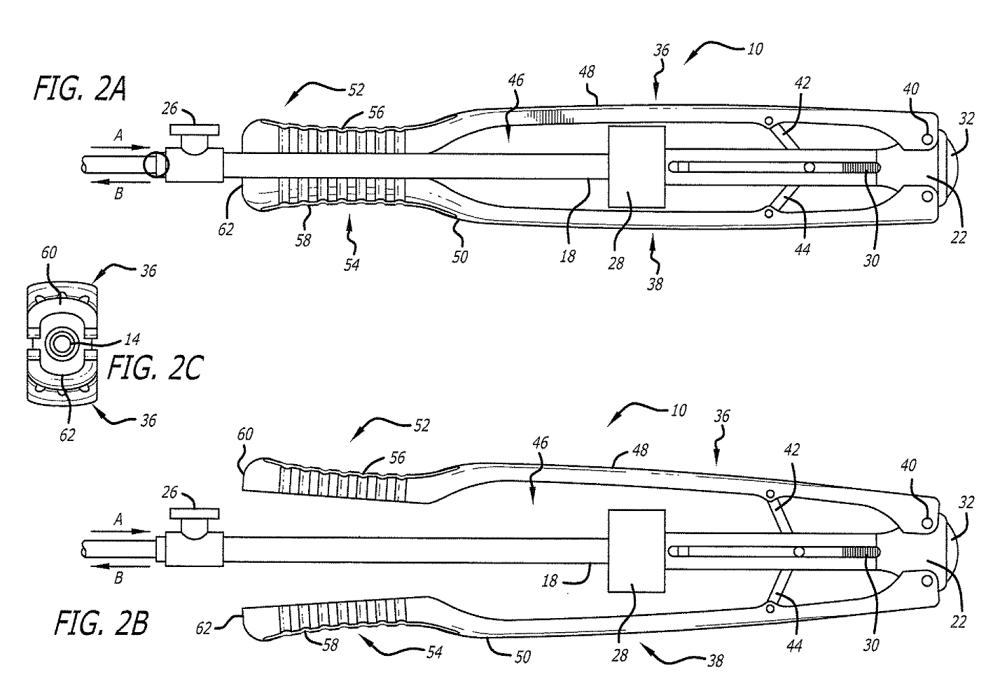

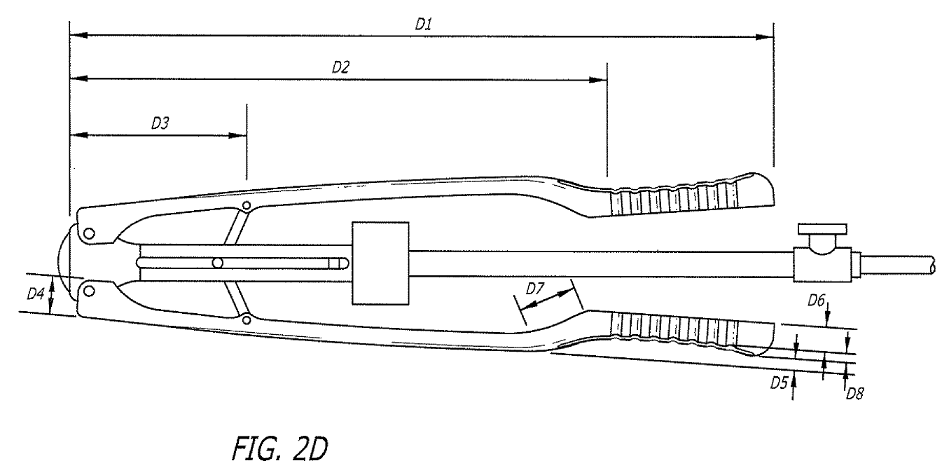

FIGS. 2A-2C illustrate the handle 10 and surgical instrument 12 of the embodiment illustrated in FIG. 1 in greater detail. As illustrated, the surgical instrument 12 includes an instrument housing 22 connected to the second end 18 of the shaft 14. The housing includes an interface connector 28 for connection to the shaft. The housing further includes a bias mechanism 30 connected to the end of the rod 24. The bias mechanism biases the rod 24 toward the first end 16 of the shaft 14. The bias mechanism is typically a compression spring, but may alternatively be any other bias mechanism suitable for biasing the rod 24. In this embodiment, the bias mechanism 30 includes an end cap 32 connected to the housing 22 and a spring 34 located between the end cap and the end of the rod to bias the rod in a direct B.

As illustrated, the handle 10 of this embodiment includes first and second lengthwise extending handle portions, respectively 36 and 38. The handle portions 36, 38 are in an opposed relationship and are pivotally connected to the instrument housing 22 at a distal position B. In the illustrated embodiment, the handle portions are connected to the housing via pivot pins 40. In other embodiments, the handle portions may be connected via living hinges to the housing 22. The first and second handle portions are also mechanically connected to the rod 24 of the surgical instrument. In the illustrated embodiment, the handle 10 includes first and second linkages, respectively 42 and 44. The first linkage 42 is pivotably connected to both the first handle portion 36 and the rod 24, and the second linkage 44 is pivotably connected to both the second handle portion 38 and the rod 24.

In this embodiment, the first and second linkages are respectively connected to the first and second handle portions at a position C and to the rod at a position D. In this embodiment, the position D is more distal from the connection of the rod to the working end 16 than the position C where the linkages 42 and 44 are respectively connected to the handle portions 36 and 38. In this configuration, when the two handle portions 36 and 38 are moved toward one another, such as when squeezed together by a user's hand (See FIG. 2A), the rod 24 is moved in a direction B, which causes the cutting edges 20 of the scissors to move toward each other in a cutting motion. In this embodiment, the bias mechanism 30 biases the handle portions 36 and 38 away from each other so that when the handle portions are not being moved inwardly toward each other, such as by squeezing, the handles maintain the cutting edges 20 in an open position. (See FIG. 2B).

As understood, FIGS. 2A-2C only illustrate one embodiment of the handle of the present invention. For example, in some instances, not shown, the rod 24 of the surgical instrument 12 may be configured relative to the working end 16 such that the rod is biased in a direction A away from the working end. In this embodiment, the first and second linkages 42 and 44 may be respectively connected to the first and second handle portions 36 and 38 at a position C and to the rod at a position D. In this embodiment, the position C where the linkages 42 and 44 are respectively connected to the handle portions 36 and 38 is more distal from the connection of the rod to the working end 16 than the position D where the linkages 42 and 44 are connected to the rod. In this configuration, when the two handle portions 36 and 38 are moved toward one another, such as when squeezed together by a user's hand, the rod 24 is moved in a direction A, which causes the cutting edges 20 to move toward each other in a cutting motion.

FIGS. 2A-2C also illustrate that the first and second handle portions 36 and 38 are connected to a distal end E of the housing 22 and extend in a direction toward the working end 16 of the surgical instrument 12. While not shown, it is understood that in an alternative embodiment, the handle portions 36 and 38 may be pivotably connected to the instrument housing 22 at a position more proximal to the working end 16 of the surgical instrument 12. In this embodiment, the first and second handle portions 36 and 38 would extend distally away from the working end 16 of the surgical instrument. In this embodiment, the first and second linkages 42 and 44 would be appropriately connected to the first and second handle portions and the rod to move the rod in an appropriate direction A and B to actuate the working end 16.

Referring again to FIGS. 2A-2C, the first and second handle portions 36 and 38 of one embodiment have a reduced side profile. This reduces the amount of surface area making the handle lighter and also providing less surface area requiring cleaning and sanitizing. The reduced side profiles also allow for access to the first and second linkages 42 and 44 and their respective pivot connections to the first and second handle portions 36 and 38 for purposes of cleaning via opening 46. As illustrated, in some embodiments, the instrument housing 22 may also have an opening 45 providing access to the distal end of the rod 24 and the connections of the first and second linkages 42 and 44 to the rod for better cleaning.

In some embodiments, the first and second handle portions 36 and 38 may have respective outer contoured surfaces 48 and 50 to provide an ergonomic design for fitting in a user's hand. As illustrated, the contoured surfaces 48 and 50 may include narrow portions 52 and 54 for placement of a user's finger or thumb. In some embodiments, the contoured surfaces 48 and 50 may include gripping surfaces 56 and 58. The gripping surfaces may be any desired size and at any desired location for improving desired gripping of the handle.

With reference to FIG. 2C, an end view of the first and second handle portions 36 and 38 is provided. In some embodiments, the first and second handle portions 36 and 38 may include arched or angled inner surfaces 60 and 62. In the embodiment of FIG. 2C, the inner surfaces 60 and 62 each have a hollow semi-elliptical profile. These inner surfaces allow the first and second handle portions 36 and 38 to have added clearance relative to the shaft 14 of the surgical instrument 12. These arched or angled inner surfaces, in turn, allow the handle to have a more compact profile. It also provides added control of the surgical instrument.

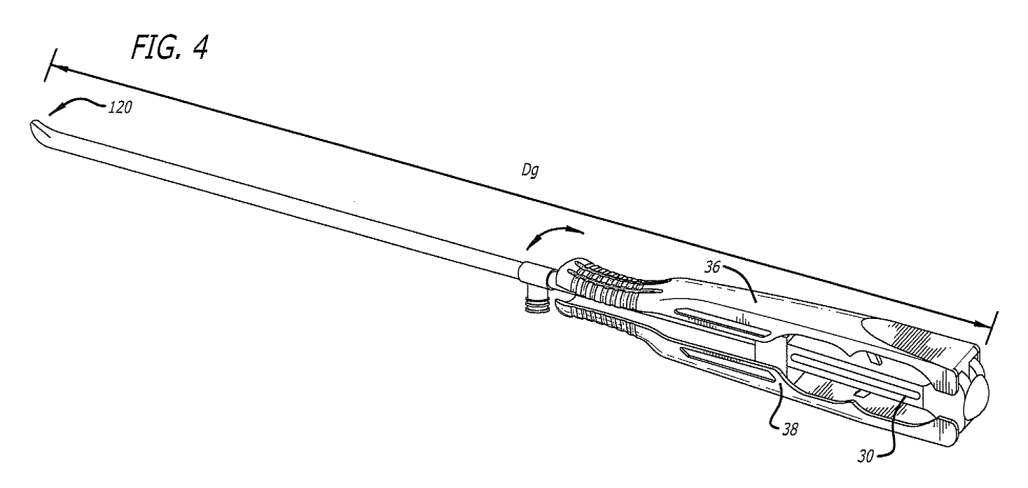

With reference to FIG. 2D, one embodiment of the invention is depicted. This embodiment illustrates a version of the handle with approximate dimensions for various elements of the handle. It is understood that this is only one embodiment of the invention and that the invention in general is not limited to specific dimensions. But an embodiment having approximately the following dimensions has been found to be particularly versatile in a variety of minimally invasive procedures in which the surgeon requires precise control of the instrument. In the embodiment of FIG. 2D, D1 is 11.3 cm., D2 is 8.5 cm., D3 is 2.6 cm, D4 is 0.6 cm., D5 is 0.6 cm., D6 is 1.0 cm., D7 is 0.8 cm., and D8 is 0.2 cm. The total length of this embodiment of the device, measuring to the end of the working end in the manner illustrated in FIG. 4, is approximately 35 cm. It has been determined that an embodiment of the handle having the dimensions described above is particularly advantageous for use by a wide variety of surgeons, permitting a single-sized handle to be employed in multiple applications, thereby reducing production and other costs associated with making several different handle sizes.

Two relevant ratios pertaining to the dimensions are as follows. The total of the dimensions D5+D6+D8 is 1.8 cm. The inward dimension of the indented grasping area is D5+D8=0.8 cm. Consequently, the ratio of the depth of the indentation to the depth of the full handle at that point is (D5+D8)/(D5+D6+D8)=0.8 cm/1.8 cm=0.44. This may be referred to, in the context of this design, as the “indentation depth ratio.” Another relevant ratio relates to the total length of a handle portion D1 relative to the distance to the proximal end of the gripping area, D2. In this embodiment, D2/D1−8.5/11.3=0.75, which is referred to as the “indentation length ratio.” Generally, in this particularly well-performing embodiment, an indentation depth ratio of between 0.35 and 0.55 is desirable, and an indentation length ratio of between 0.65 and 0.85 is desirable, in order to facilitate desirable control and ergonomic features of the device.

As mentioned above, a biasing mechanism 30 is located in communication with the rod 24 to bias the rod. In some embodiments, the biasing mechanism may be configured to provide added control between the handle 10 and the working end 16. The biasing mechanism can be designed to allow for variability of the force necessary to activate the working end 16, in this case, the cutting edges 20.

For example, where a spring is used as the biasing mechanism, a more compliant spring (i.e., less resistive) will reduce the activation force, while a less compliant spring will increase the activation force. The specification of the spring could be chosen during the manufacturing process or it could be selected by the user. In this latter instance, user selection could be made by allowing the user to replace the spring by selecting from a plurality of replacement springs having different tensioning. In another or additional embodiment, the biasing mechanism may include an adjustable bias aspect that allows the user to alter biasing when desired or needed. For example, the biasing mechanism of this embodiment may include a spring with screw tension adjustment to set and adjust the tension on the spring.

The biasing system may also offer the benefit of limiting the force that forceps, scissors and needle drivers close and open. This “terminal force” may be a benefit, for example, if the surgeon has a piece of tissue in the forceps and depresses the handle more. Even though the handles are pushed further down, the forceps will only exert a maximum force as specified by the spring chosen. Here again, the amount of bias may be selected and/or adjusted based on the embodiment.

The ergonomic and compact design of the handle allows the instrument to be more easily manipulated. For example, because the first and second handle portions 36 and 38 of some embodiments are located in compact adjacent relationship to the shaft, the handle may allow the surgical instrument to be more easily rotated in the hands of the user without the need to remove the surgical instrument from the surgical site to be repositioned in the user's hand. Further, the compact design may allow for use of the surgical instrument in close proximity to other surgical instruments without less obstruction. The ergonomic design of some embodiments may also allow for increased control of the surgical instrument. Due in part to the simply mechanical linkages used, the arched or angled inner surfaces 60 and 62 that allow for greater clearance of the first and second handle portions 36 and 38 relative to the shaft, and the ergonomic design, the handle may achieve at or near 1 to 1 ratio between action of the user's hands during activation and the action of the working end of the instrument, which allows the instrument to feel as though it is an extension of the user's hand.

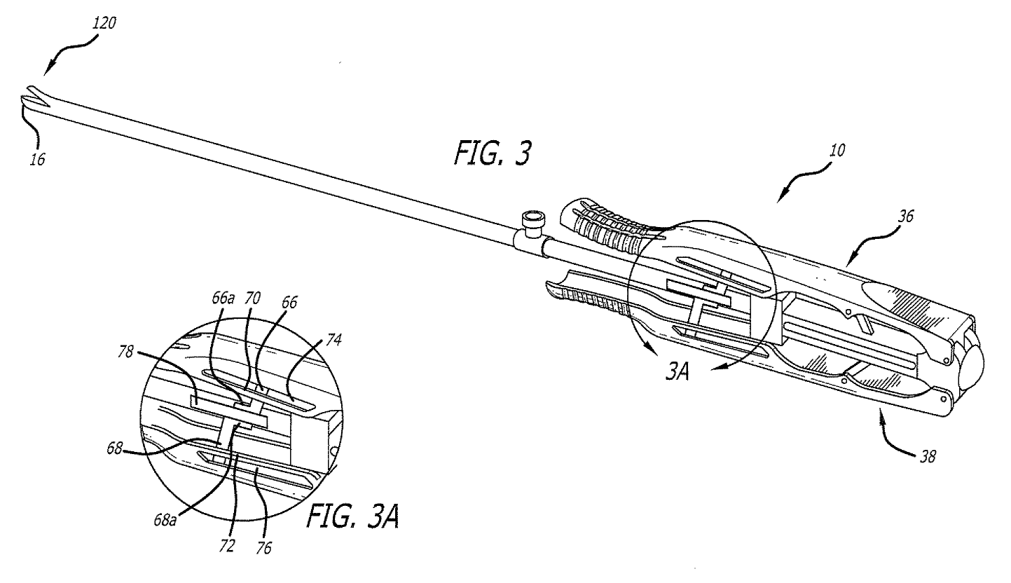

FIGS. 3-7 illustrate another embodiment of the handle 10 of the present invention. In this embodiment, the working end 16 of the surgical instrument 12 includes a grasper or clamp. One use of the embodiment of FIGS. 3-7 is to hold a needle for suturing inside the body. The gripping portion 120 is shown in FIG. 3 in an open configuration in which the respective handle portions are themselves in an open position to which they are normally biased.

When clamps or similar surgical instruments are employed, it may be beneficial to lock the clamps in place during use, as illustrated in FIG. 4. For this reason, this embodiment of the handle 10 further includes a locking mechanism 64 (FIG. 3A). The locking mechanism 64 of this embodiment includes first and second locks 66 and 68 respectively connected to the first and second handle portions 36 and 38. The first and second locks 66 and 68 respectively include L-shaped portions 66a and 68a. The L-shaped portions have curved surfaces extending laterally. At least one of the L-shaped portions comprises a laterally extending flange. The flange operates to couple the two L-shape portions 66a and 68a when the first and second locks 66 and 68 are brought into contact with each other when the first and second handle portions 36 and 38 are squeezed or otherwise moved toward each other, thereby locking the handle portions in place and the working end at a fixed position.

In the illustrated embodiment, the locks 66 and 68 are somewhat centrally located widthwise in the handle. To accommodate for this placement, the surgical instrument of the illustrated embodiment includes a slot 78 in the shaft to provide clearance for the locks. While not illustrated, in some embodiments, the first and second locks could be placed in an off-center position in the handle so as to avoid the shaft.

As illustrated, the first and second locks 66 and 68 may be coupled respectively to the first and second handle portions 36 and 38 so as to be laterally moveable relative to the first and second portions. Specifically, in one embodiment, the first and second locks 66 and 68 are located in slots 70 and 72 respectively in the first and second handle portions 36 and 38. The first and second locks are laterally biased by bias mechanism 74 and 76, such as springs. In this configuration, when the first and second handle portions 36 and 38 are move toward each other, the L-shaped portions 66a and 68b of the first and second locks 66 and 68 are brought into engagement with other to interlock. However, further movement of the first and second handle portions relative to each other will cause the biasing mechanism to move the first and second locks away from each other laterally. It is understood that FIGS. 3 and 3A illustrate only one embodiment of a locking mechanism that may be employed with the handle of the present invention.

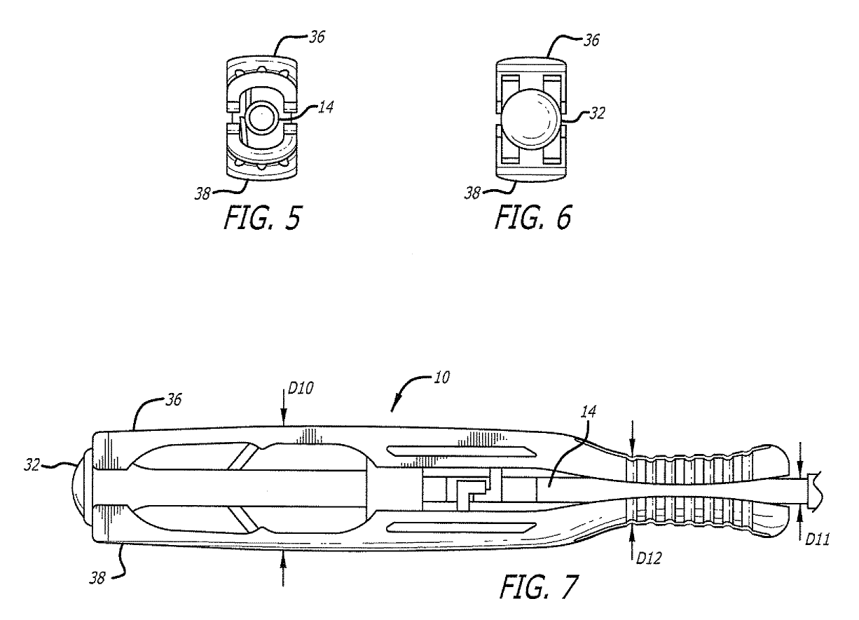

FIG. 4 illustrates the embodiment of FIG. 3 in a closed configuration. The gripper portion 120 is closed and can hold, for example, a needle used in suturing. The cross-section of the indented portions of the handle may be, for example, semi-elliptical (see, e.g., the profile illustrated in FIG. 5). It has been found by the inventors that a semi-elliptical profile assists the surgeon during surgery in that by moving a finger and a thumb, for instance, the surgeon may smoothly rotate the device. This is of significant advantage in suturing the patient, since the device must be rotated in one or both directions during suturing.

FIG. 6 illustrates the end cap 32 which, as previously described, may work in conjunction with a compression spring or other biasing device to bias the handle and the working end into an open configuration. As the biasing device may take alternative forms, the end cap 32 is part of a presently preferred embodiment, but may be unnecessary in other embodiments.

In one embodiment of the present invention, the handle provides rotational stability by having, for example, a portion that is considerably wider than the shaft. This width helps to create rotational inertia, which tends to resist rotation of the shaft. The device is therefore more stable at times when the surgeon does not want the device to rotate, and helps to slow the rotation as the surgeon is rotating the device. This gives the surgeon better control during the delicate stitching process. In FIG. 7, D10 is a width of the handle at a wide point (in either of the embodiments of FIGS. 1-3 or 4-6). D11 is the diameter of the shaft 14. In one embodiment, the ratio of the handle width D10 to the width or diameter of the shaft 14 is about 2.2 cm to 0.5 cm, or a ratio greater than 4:1. In the embodiment of FIG. 7, this ratio creates desirable and advantageous rotational control properties.

Additionally, the width of the handle in the closed configuration at a narrow portion at the base of an indented portion, D12, is about 1.1 cm in the embodiment of FIG. 7. This results in a ratio of about 2:1 between the width at a widest portion of the handle 10 to the width at the narrowest portion. The indented portion is thereby able to accommodate a finger and a thumb of the surgeon.

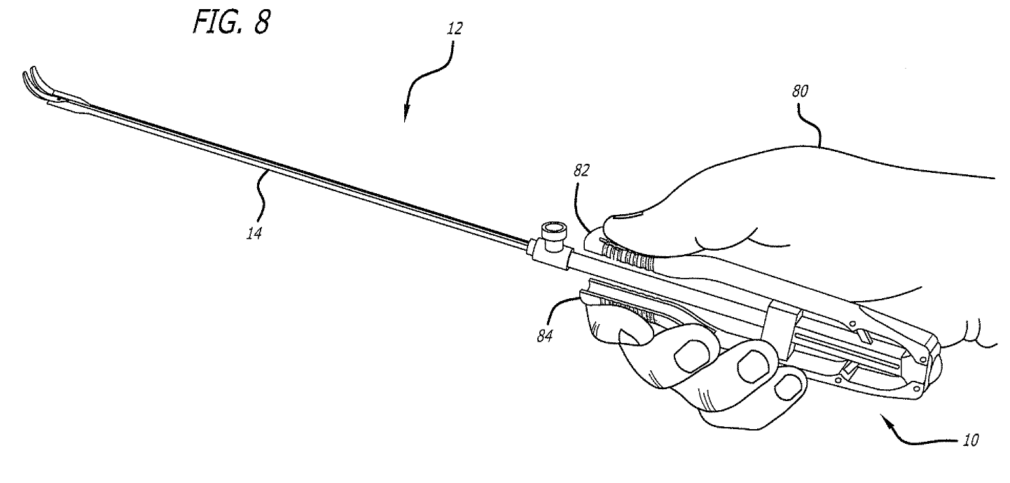

Various aspects of the handle construction provide added functionality and ease of use. As a few non-limiting examples, the indented portion of the handle allows the surgeon to quickly locate the proper location on the handle to grasp. The round and/or semi-elliptical cross-section at the indented portion provides the surgeon with the ability to smoothly rotate the device during surgery. In some embodiments, the width of the handle at a wider portion creates rotational inertia that provides rotational stability. The relative dimensions of certain embodiments and/or the relative weight of the handle in relation to the shaft, balances the device for improved control. In one embodiment, the handle has two portions. The proximal portion is relatively heavy. The distal portion curves inward sharply, has a curved cross-section and is lighter than the proximal portion. A surgeon grasps the handle at the distal, inwardly curved portion with the thumb and a finger, and holds the device as in FIG. 8 or, alternatively, like holding a pen.

Removal of non-essential and non-functional elements of the handle in some embodiments minimizes the mass and ultimate weight of the handle. Use of contoured handle elements and gripping elements in some embodiments provides a natural resting place for fingers and aids in handle manipulation. The open handle design of some embodiments allows for easy cleaning of the device that may otherwise be hidden and not easily verifiable as clean. Also, the compact nature of the handle and circular gripping area allow for easy manipulation and rotation of the surgical instrument with reduced requirements for repositioning of the instrument. First and second handle portions 36, 38 and other components such as interface connector 28 may be made of titanium, which has properties of strength and light weight. The shaft 14 is typically made of stainless steel, although alternative materials may be used for both the handle 10 and the shaft 14.

Minimizing connection points between the handle and the shaft minimizes jamming and/or malfunctioning of the surgical instrument. Also, the linkage used in some embodiments of the design allow for 1 to 1 ratio or near 1 to 1 ratio between action of the user's hands during activation and the action of the working end of the instrument, which allows the instrument to feel as though it is an extension of the user's hand. The use of titanium in some embodiments increases the stability and longevity of the handle, decreases weight, and eliminates most handle degradation due to use and cleaning.

FIG. 8 illustrates a surgeon's hand 80 grasping a device according to the present invention, prior to insertion into the body during surgery. The thumb and first finger grasp the handle near the distal ends 82, 84 of the first and second portions, respectively, of handle 10. The balance point of the device of the embodiment of FIG. 8 is at the distal ends 82, 84 of the handle. Consequently, the device provides the surgeon with enhanced control during surgery.

In a preferred embodiment, the device has a balance point at the distal end of the handle, when the handle is in a closed configuration. Consequently, when the surgeon grasps the handle at the indentations, as illustrated in FIG. 8, the finger and thumb rest at approximately the balance point of the device. This “balance point” is the point along the device at which there is equilibrium between proximal and distal portions of the device. This location of the balance point, in conjunction with the narrow indented grasping portions 52, 54 (FIG. 1) in which the surgeon's fingers and/or thumb are nested, assists the surgeon by making the device stable in use, and enhances the surgeon's ability to precisely perform medical procedures such as cutting, grasping and/or suturing at locations in the body remote to the surgeon. As an alternative to FIG. 8, the surgeon may choose to hold the instrument like a pen, with the index finger pointing toward the distal end of the handle, in order to easily rotate the instrument.

Referring to the specific, non-limiting embodiment of FIGS. 2D and 4, the balance point is at about a distance D1 from the proximal end of the device. In one specific embodiment, distance D1 is D1/D9=11.3 cm/35 cm=0.32, or at roughly ⅓ of the total length of the device. More generally, a presently preferred embodiment of the device is weighted in the handle and, more particularly, in the proximal portion of the handle in order to create the inertia necessary to put the balance point in the proximal half of the device. Several other advantages of particular embodiments are described in the foregoing.

While particular forms of the invention have been illustrated and described, it will be apparent that various modifications can be made without departing from the spirit and scope of the invention. Accordingly, it is not intended that the invention be limited, except as by the appended claims.

What is claimed is:

1. An instrument for minimally invasive surgery comprising:
a shaft;
a rod located in the shaft and extending between a first end connected to a working end of the surgical instrument and a distal second end for connecting to the handle;
wherein the handle has:
first and second handle portions that are in an opposed relationship and that are in pivotal communication with the shaft; and
first and second linkages respectively connecting the first and second handle portions to the rod at or near the distal end of the rod, wherein when the first and second handles are moved relative to each other about their respective pivot points relative to the shaft, the rod operates the working end of the surgical instrument; and
wherein the first and second handle portions each have an indented grasping segment in a distal region of each handle portion.

2. An instrument as defined in claim 1, wherein the indented grasping segment comprises a rounded cross-section to facilitate a surgeon rotating the handle with fingers.

3. An instrument as defined in claim 1, wherein each grasping segment includes gripping surfaces comprising ridges.

4. An instrument as defined in claim 1, wherein the handle has an open configuration and a closed configuration, and the indented portions together form a narrow handle portion when the handle is in the closed configuration.

5. An instrument as defined in claim 1, wherein each of the indented segments has a semi-elliptical cross-section.

6. An instrument as defined in claim 1, wherein the handle at a widest portion is at least two times wider than at a narrowest portion when the handle is in a closed configuration.

7. An instrument as defined in claim 1, wherein the handle at a widest portion is at least four times wider than the shaft.

8. An instrument as defined in claim 1, wherein the handle comprises walls having elongated openings to facilitate cleaning of the handle.

9. An instrument as defined in claim 1, wherein each of the indented portions includes a finger stop at a distal end thereof.

10. An instrument as defined in claim 1, wherein the handle includes a 1:1 movement ratio between handle movement and movement of the working end.

11. An instrument as defined in claim 1, wherein the balance point of the instrument is at approximately the distal end of the handle.

12. An instrument as defined in claim 1, wherein the balance point is at approximately ⅓ of the length of the instrument from the proximal end of the instrument.

13. An instrument as defined in claim 1, wherein the handle is no more than approximately one third as long as the total length of the surgical instrument.

14. An instrument as defined in claim 1, wherein the indentation depth ratio is between 0.35 and 0.55.

15. An instrument as defined in claim 1, wherein the indentation length ratio is between 0.65 and 0.85.

16. An instrument as defined in claim 1, wherein the handle has a relatively heavier proximal portion and a relatively lighter distal portion that sharply curves inwardly, wherein the relatively heavier proximal portion acts as a counterweight to the shaft.

17. An instrument for minimally invasive surgery comprising:
a shaft;
a rod located in the shaft and extending between a first end connected to a working end of the surgical instrument and a distal second end for connecting to the handle, wherein the handle has:
first and second handle portions that are in an opposed relationship and that are in pivotal communication with the shaft; and
first and second linkages respectively connecting the first and second handle portions to the rod at or near the distal end of the rod, in which when the first and second handles are moved relative to each other about their respective pivot points relative to the shaft, the rod operates the working end of the surgical instrument;
the first and second handle portions each having an indented grasping segment in a distal region of each handle portion;
the indented grasping segment having a rounded cross-section to facilitate a surgeon rotating the handle with fingers;
the handle having an open configuration and a closed configuration, and the indented portions together forming a narrow rounded handle portion when the handle is in the closed configuration; and
each of the indented portions includes a finger stop at a distal end thereof.

18. An instrument as defined in claim 17, wherein each grasping segment includes gripping surfaces comprising ridges.

19. An instrument as defined in claim 17, wherein each of the indented segments comprises a semi-elliptical cross-section.

20. An instrument as defined in claim 17, wherein the handle at a widest portion is at least four times wider than at a narrowest portion when the handle is in a closed configuration.

21. An instrument as defined in claim 17, wherein the handle comprises walls having elongated openings to facilitate cleaning of the handle.

22. An instrument as defined in claim 17, wherein the handle includes a 1:1 movement ratio between handle movement and movement of the working end.

23. An instrument as defined in claim 17, wherein the handle is no more than approximately one third as long as the total length of the surgical instrument.

24. An instrument as defined in claim 17, wherein the balance point of the instrument is at approximately the distal end of the handle.

25. An instrument as defined in claim 17, wherein the balance point is at approximately 113 of the length of the instrument from the proximal end of the instrument.

26. An instrument for minimally invasive surgery comprising:
a shaft;
a rod located in the shaft and extending between a first end connected to a working end of the surgical instrument and a distal second end for connecting to the handle, the handle comprising:
first and second handle portions that are in an opposed relationship and that are in pivotal communication with the shaft; and
first and second linkages respectively connecting the first and second handle portions to the rod at or near the distal end of the rod,
whereby when the first and second handles are moved relative to each other about their respective pivot points relative to the shaft, the rod operates the working end of the surgical instrument; and wherein:
the first and second handle portions each have an indented grasping segment in a distal region of each handle portion;
the indented grasping segment comprises a rounded cross-section to facilitate a surgeon rotating the handle with fingers;
each grasping segment includes gripping surfaces comprising ridges;
the handle has an open configuration and a closed configuration, and the indented portions together form a narrow handle portion when the handle is in the closed configuration each of the indented segments comprises a semi-elliptical cross-section;
the handle at a widest portion is at least two times wider than at a narrowest handle portion when the handle is in a closed configuration;
the handle comprises walls having elongated openings to facilitate cleaning of the handle;
each of the indented portions includes a finger stop at a distal end thereof;
the handle includes a 1:1 movement ratio between handle movement and movement of the working end;
the indentation depth ratio of the handle is between 0.35 and 0.55;
the indentation length ratio of the handle is between 0.65 and 0.85; and
the balance point of the instrument is at approximately the distal end of the handle.

27. An instrument as defined in claim 26, wherein the handle is no more than approximately one third as long as the total length of the surgical instrument.

28. An instrument as defined in claim 26, wherein the handle is made of titanium.

29. An instrument as defined in claim 26, wherein the ratio of the handle width to the diameter of the shaft is at least 4:1.

30. An instrument as defined in claim 26, wherein the balance point is at approximately ⅓ of the length of the instrument from the proximal end of the instrument.

31. An instrument as defined in claim 26, wherein the handle has a relatively heavier proximal portion and a relatively lighter distal portion that sharply curves inwardly, wherein the relatively heavier proximal portion acts as a counterweight to the shaft.
